(19) United States
(12) Patent Application Publication
(10) Pub. No.: US 2024/0033060 A1
(43) Pub. Date: Feb. 1, 2024

(54) ARTIFICIAL INTELLIGENCE-BASED METHOD AND APPLICATION FOR MANUFACTURING 3D PROSTHESIS FOR TOOTH RESTORATION

(71) Applicants: Hyuk Jun PARK, Dongdaemun-gu Seoul (KR); Sung Gyeol LIM, Hwaseong-si Gyeonggi-do (KR); Ji Woong YU, Dongdaemun-gu Seoul (KR); Seung Hyeon HAM, Yangpyeong-gun Gyeonggi-do (KR)

(72) Inventors: Hyuk Jun PARK, Dongdaemun-gu Seoul (KR); Sung Gyeol LIM, Hwaseong-si Gyeonggi-do (KR); Ji Woong YU, Dongdaemun-gu Seoul (KR); Seung Hyeon HAM, Yangpyeong-gun Gyeonggi-do (KR); Young Kwang NAM, Eunpyeong-gu Seoul (KR)

(21) Appl. No.: 18/276,706

(22) PCT Filed: Oct. 20, 2021
(86) PCT No.: PCT/KR2021/014715

§ 371 Date: Aug. 10, 2023

(30) Foreign Application Priority Data
Feb. 16, 2021 (KR) 10-2021-0020702

(51) Int. Cl. A61C 13/00; A61C 13/34; G06F 30/27; G06V 10/772; G06V 10/82

(52) U.S. Cl. CPC A61C 13/0004; A61C 13/34; G06F 30/27; G06V 10/772; G06V 10/82; G06V 2201/03

(57) ABSTRACT

The present invention relates to an artificial intelligence-based method for manufacturing a 3D prosthesis for tooth restoration, the method including the steps of (a) obtaining an image of arrangement of teeth including an abutment tooth and a 3D modeling image of a crown which is a prosthesis, (b) preprocessing the 3D modeling image of the abutment tooth and the 3D modeling image of the crown into a 2D image so as to establish a learning dataset, (c) performing first artificial intelligence learning by using an artificial intelligence image conversion algorithm with the abutment tooth image and the crown image having been established into the learning dataset as reference data and a crown image generated appropriately to be intubated into the abutment tooth considering a shape and a size of a peripheral tooth of the abutment tooth as a correct answer data, and (d) performing second artificial intelligence learning for extracting a 3D learning image of a crown in which a volume is assigned to the 2D learning image of the crown by using the artificial intelligence image conversion algorithm on the basis of shape information and depth information of the 2D learning image of the crown and the 2D learning image of the abutment tooth extracted by the first artificial intelligence learning.

### TECHNICAL FIELD

The present invention relates to a manufacturing method and application for automatically designing a prosthesis, and more particularly, to a method and application for manufacturing a prosthesis that automatically designs a 3D image of a crown suitable for an arrangement of teeth of a patient based on an artificial intelligence algorithm.

### BACKGROUND ART

Impression taking in a process of manufacturing a dental prosthesis is a clinical process that is the basis for diagnosis of the patient, establishing future treatment plans, or manufacturing a patient-specific prosthesis by applying the condition of teeth and tissues in an oral cavity to impression materials. A general impression taking method requires skilled clinical skills of an operator.

Accordingly, research is being conducted to utilize a computer for the design or processing of the dental prosthesis, which is performed manually, and to automate the design and production of the prosthesis. Conventionally, the oral cavity is digitally scanned using an intraoral scanner, a scanned 3D image of an arrangement of teeth is modeled and displayed, and then the dental prosthesis is designed with a computer based on the modeled image of the arrangement of teeth. Here, a recent prosthesis design system analyzes the oral cavity image through an advanced image analysis tool and provides oral information useful for designing the prosthesis through a graphical user interface (GUI) tool. In this regard, there is Korean Patent Registration No. 10-1994396.

However, in the prosthesis design system, the prosthesis is still designed through drawing, which is manual work, and design quality of the prosthesis depends on an ability of the operator and takes excessive work time, and thus a professional designer is separately employed.

With this background, there is a need for automation of tooth design in a prosthesis manufacturing CAD program which is currently in use. Until now, the reason why drawing, which is manual work, is required when designing the prosthesis is that a lost crown area should be designed in a shape suitable for the arrangement of teeth of the patient. In this case, the operator draws manually so as to properly match the peripheral tooth in consideration of dental conditions of the patient, a size of adjacent teeth, a groove, etc. and a variety of GUIs have recently been provided to assist the manual drawing, but a professional operator is still required.

On the other hand, currently, an open source of artificial intelligence algorithm is widely distributed and the application of artificial intelligence technology is being grafted into various fields, and a technology that automatically generates a crown by designing customized teeth according to the patient by machine learning through an AI algorithm is being developed. Korean Patent Registration No. 10-2194777 discloses a design system that automatically designs a dental prosthesis through artificial intelligence-based data learning. The prior literature discloses a solution for assigning an evaluation score to work data on teeth, classifying and storing the work data in order to automatically design a prosthesis of a certain level regardless of the operator's skill level, and performing an appropriate dental prosthesis design based on the similarity between tooth data and the evaluation score from a server when a request for dental prosthesis is received. Prior literature of Korean Patent Registration No. 10-2194777 discloses an application example of artificial intelligence in the direction of recommending the most suitable model for the prosthesis requested based on a large number of pieces of basic work data.

However, in this case, teeth big data of infinitely various shapes and sizes of the arrangement of teeth may be required. In order to design a design suitable for the arrangement of teeth of the patient, a process of training artificial intelligence to create the shape itself by the artificial intelligence itself considering the shape of the peripheral tooth can be a more fundamental problem to be solved.

Therefore, the present applicant has applied for an invention of forming a crown similar to adjacent teeth by applying a GAN artificial intelligence-based algorithm in Application No. 10-2020-0024114. However, in the prior application, a 3D scanned tooth image was learned on a 2D image to extract a shape of the crown suitable for the 2D image, but a technical problem to be solved of modeling the 3D crown applicable in the actual field remained.

Expanding the 2D image of the crown to 3D in this technical field cannot be solved with a task of 3D modeling that simply gives volume. More complex practical issues should be considered for this expansion. The shape of the appropriate tooth image of the crown derived by artificial intelligence is the design of an upper surface (top surface) in contact with an antagonist tooth. When expanding it to 3D, the following technical problems arise. First, a lateral surface (circumferential surface) of the tooth should also be designed with appropriate curves and shapes. Second, since the crown is intubated into and covered on an abutment tooth (abutment), the design of an inner groove thereof to be intubated into the abutment tooth is required, which requires consideration of various shapes of the abutment tooth for each patient. Third, the crown should be designed to fit a margin line set by the operator.

For this reason, when learning an image of 2D tooth data, not only the design elements that match the peripheral tooth, but also information of the abutment tooth should be learned together, and accordingly, the present applicant has devised a method and application for automatically performing final 3D crown modeling by additionally performing artificial intelligence learning by reflecting depth information in the process of deriving 2D crown information suitable for the peripheral tooth and abutment tooth and expanding it to 3D.

### PRIOR ART LITERATURE

### Patent Literature

Korean Patent Registration No. 10-1994396
Korean Patent Registration No. 10-2194777

### DISCLOSURE OF THE INVENTION

### Technical Problem

The present invention aims to provide an artificial intelligence-based method and application for manufacturing a prosthesis for tooth restoration that automatically produces a crown image required for prosthesis design as an image suitable for teeth of a patient based on learning information of a deep learning algorithm.

In addition, the present invention aims to provide an artificial intelligence-based method and application for manufacturing a prosthesis for tooth restoration that produces a 3D crown image so that an internal volume that can be inserted into an abutment tooth can be formed by reflecting abutment tooth data of a patient.

### Technical Solution

In order to achieve the above object, according an aspect of the present invention, there is provided an artificial intelligence-based method for manufacturing a 3D prosthesis for tooth restoration, the method including the steps of (a) obtaining an image of arrangement of teeth including an abutment tooth and a 3D modeling image of a crown which is a prosthesis, (b) preprocessing the 3D modeling image of the abutment tooth and the 3D modeling image of the crown into a 2D image so as to establish a learning dataset, (c) performing first artificial intelligence learning by using an artificial intelligence image conversion algorithm with the abutment tooth image and the crown image having been established into the learning dataset as reference data and a crown image generated appropriately to be intubated into the abutment tooth considering a shape and a size of a peripheral tooth of the abutment tooth as a correct answer data, and (d) performing second artificial intelligence learning for extracting a 3D learning image of a crown in which a volume is assigned to the 2D learning image of the crown by using the artificial intelligence image conversion algorithm on the basis of shape information and depth information of the 2D learning image of the crown and the 2D learning image of the abutment tooth extracted by the first artificial intelligence learning.

Preferably, in the step (a), an arrangement of teeth image including at least one tooth on the left and right of the abutment tooth may be used as a modeling target as the image of the arrangement of teeth for which 3D modeling is performed.

Preferably, in the step (b), the 3D modeling image of the abutment tooth and the 3D modeling image of the crown may be pre-processed into a 2D image obtained by photographing cross-sections of the 3D modeling image with a certain angle as a reference.

Preferably, in the step (b), the 3D modeling image of the abutment tooth and the 3D modeling image of the crown may be pre-processed into a 2D image obtained by photographing the 3D modeling images under different lighting.

Preferably, in the step (b), a 2D modeling image of an antagonist tooth or a margin line image of the abutment tooth may be included in the learning dataset.

Preferably, in the step (b), a 2D image may be generated with 4 channels of an RGB image (3ch) and an upper surface depth image (1ch) of a tooth.

Preferably, in the step (c), an artificial intelligence image conversion algorithm of a neural network model utilizing an image encoder decoder may be used.

Preferably, in the step (c), learning of a first-stage model with the 2D modeling image of the abutment tooth as reference data and the 2D modeling image of the crown as correct answer data and a second-stage model with a result of the first-stage model as reference data and the 2D modeling image of the crown as correct answer data may be performed by the first artificial intelligence learning.

Preferably, in the step (d), a three-dimensional reconstruction algorithm for composing a 2D image into a 3D image may be applied as a neural network model utilizing an image encoder decoder.

Preferably, in the step (d), a 3D learning image of the crown may be generated based on an RGB-D 4-channel image of the abutment tooth and an RGB-D 4-channel image of the crown extracted by the first artificial intelligence learning.

Further, according another aspect of the present invention, there is provided an artificial intelligence-based application for manufacturing a 3D prosthesis for tooth restoration, the application being stored in a medium in order to cause a smartphone, tablet, laptop, or computer having an input means for inputting data, a processing means for processing the input data, and an output means to execute the functions of obtaining an image of arrangement of teeth including an abutment tooth and a 3D modeling image of a crown which is a prosthesis, preprocessing the 3D modeling image of the abutment tooth and the 3D modeling image of the crown into a 2D image so as to establish a learning dataset, performing first artificial intelligence learning by using an artificial intelligence image conversion algorithm with the abutment tooth image and the crown image having been established into the learning dataset as reference data and a crown image generated appropriately to be intubated into the abutment tooth considering a shape and a size of a peripheral tooth of the abutment tooth as a correct answer data, and performing second artificial intelligence learning for extracting a 3D learning image of a crown in which a volume is assigned to the 2D learning image of the crown by using the artificial intelligence image conversion algorithm on the basis of shape information and depth information of the 2D learning image of the crown and the 2D learning image of the abutment tooth extracted by the first artificial intelligence learning.

### Advantageous Effects

According to the present invention, the crown image required for prosthesis design is automatically produced as an image suitable for teeth of a patient based on learning information of the deep learning algorithm, thereby capable of reducing the time and labor cost required for manual work.

In addition, according to the present invention, as learning is performed by matching the abutment teeth and crowns and the shape and depth information of the learned 2D images are additionally learned and extended to 3D, there is an advantage in that 3D modeling of the crown is possible with a volume suitable for the shape of the abutment tooth.

### BRIEF DESCRIPTION OF THE DRAWINGS

FIG. 1 illustrates a block diagram of a configuration of an artificial intelligence-based method for manufacturing a 3D prosthesis for tooth restoration according to an embodiment of the present invention.

FIG. 2 illustrates an image of an arrangement of teeth scanned by a 3D scanner and a 3D modeling image of a designed crown.

FIGS. 3a-3d illustrate 2D images of a learning dataset obtained by preprocessing the 3D modeling images.

FIG. 4 is a learning screen performed in a first-stage model of first artificial intelligence learning.

FIG. 5 is a learning screen performed in a second-stage model of the first artificial intelligence learning.

FIGS. 6a-6b illustrate a learning screen and learning result of the first artificial intelligence learning.

FIGS. 7a-7b illustrate a learning screen and learning result of second artificial intelligence learning.

FIGS. 8a-8f illustrate a processed product manufactured as a result of the second artificial intelligence learning.

### MODE FOR CARRYING OUT THE INVENTION

Hereinafter, the present invention will be described in detail with reference to the contents described in the accompanying drawings. However, the present invention is not limited or restricted by exemplary embodiments. The same reference numerals in each figure indicate members performing substantially the same function.

The objects and effects of the present invention can be naturally understood or more clearly understood by the following description, and the objects and effects of the present invention are not limited only by the following description. In addition, in describing the present invention, if it is determined that a detailed description of a known technology related to the present invention may unnecessarily obscure the gist of the present invention, the detailed description thereof will be omitted.

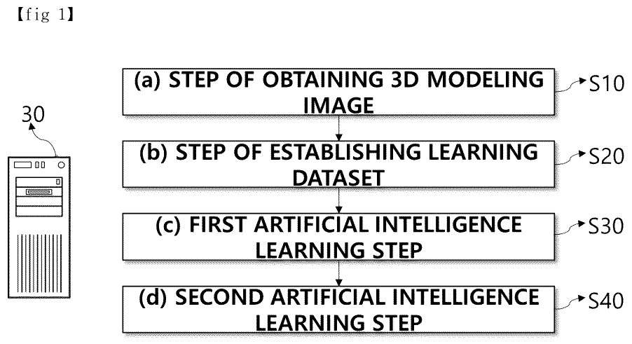

FIG. 1 illustrates a block diagram of a configuration of an artificial intelligence-based method for manufacturing a 3D prosthesis for tooth restoration according to an embodiment of the present invention.

Referring to FIG. 1, the artificial intelligence-based method for manufacturing a 3D prosthesis for tooth restoration according to an embodiment of the present invention may include (a) (S10) step of obtaining a 3D modeling image, (b) (S20) step of establishing a learning dataset, (c) step (S30) of performing first artificial intelligence learning, and (d) step (S40) of performing second artificial intelligence learning.

Steps (a) (S10) to (d) (S40) described below may be implemented as functions of an application or program stored in a medium for causing a smartphone, tablet, laptop, or computer having an input means for inputting data, a processing means for processing the input data, and an output means to execute the functions.

Steps (a) (S10) to (d) (S40) may be understood as operation steps of a program or application executed on a server 30 that performs big data implementation and artificial intelligence learning.

Step (a) (S10) is a step of acquiring an image of an arrangement of teeth including an abutment tooth and a 3D modeling image of a crown, which is a prosthesis. The 3D modeling image may be obtained from an image of arrangement of teeth of a patient scanned by a 3D scanner. It is preferable that the 3D modeling image obtained in step (a) (S10) includes the abutment tooth. The abutment tooth is a tooth that serves to support a prosthesis in the treatment planning stage before fixed or removable prosthesis treatment, and may include an abutment. The abutment tooth provides proper maintenance, support, and stability depending on a location and extent of a tooth defective part, and its shape and size are different depending on the arrangement of teeth of the patient. Above the abutment tooth, a designed crown is intubated to replace a lost tooth. Hereinafter, a crown suitable for intubation into the abutment tooth is modeled in 3D by automatically manufacturing the crown that matches the shape of the peripheral tooth and reflecting design characteristics of the abutment tooth during the automatic manufacturing of the crown.

It is noteworthy that the automatic prosthesis manufacturing process according to this embodiment proposes a learning model that learns a 3D modeling image based on 2D and expands it to 3D again. Conventionally, an artificial intelligence algorithm that learns the 3D model itself has also been disclosed, but when performing 3D-based learning, a huge amount of data and computation are required for learning the detailed shape of a tooth. Accordingly, this embodiment proposes learning the shape by converting 3D to 2D, and additionally learning so as to consider the volume and abutment characteristics in the process of expanding it to 3D.

In step (a) (S10), as the arrangement of teeth image for which 3D modeling is performed, an arrangement of teeth image including at least one tooth on the left and right of the abutment tooth is used as a modeling target.

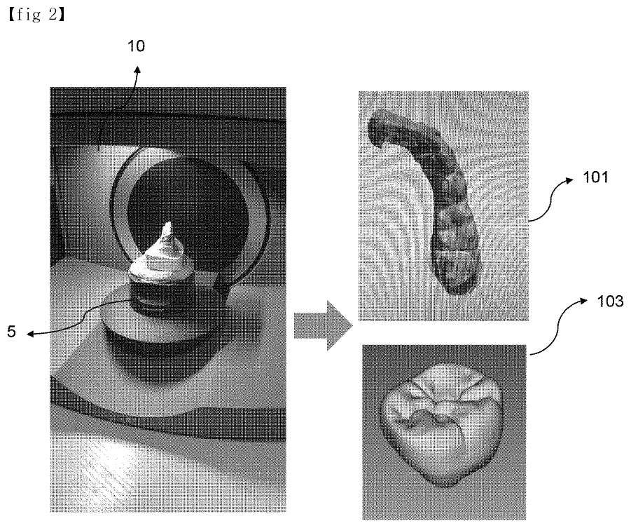

FIG. 2 illustrates an image 101 of arrangement of teeth scanned by a 3D scanner 10 and a 3D modeling image 103 of a designed crown. Referring to FIG. 2, the 3D scanner 10 scans teeth of the patient or an arrangement of teeth modeled after the teeth. In this time, an arrangement of teeth in which a designed abutment tooth is prepared is used as a scanning target, and preferably at least one tooth is included on the left and right sides of the abutment teeth. This is to learn to naturally design the shape of the crown to be covered on the abutment tooth in consideration of a shape of a peripheral tooth.

In step (a) (S10), the 3D modeling image 101 of the arrangement of teeth in which the abutment tooth is located in the middle is obtained through the 3D scanner 10. In addition, for learning, the 3D modeling image 103 of a crown in which a crown image suitable for a corresponding arrangement of teeth is produced by a worker or a dental technician is received as an input. The 3D modeling image 103 of the crown is learned through a GAN model later as correct answer data suitable for the corresponding arrangement of teeth.

Step (b) (S20) is a step of establishing a learning dataset by preprocessing the 3D modeling image 101 of the abutment tooth and the 3D modeling image 103 of the crown into a 2D image. In this embodiment, in step (b) (S20), the 3D modeling image 101 of the abutment tooth and the 3D modeling image 103 of the crown may be pre-processed into the 2D image obtained by photographing cross-sections of the 3D modeling images with a certain angle as a reference. In addition, in step (b) (S20), the 3D modeling image of the abutment tooth and the 3D modeling image of the crown may be pre-processed into the 2D image obtained by photographing the 3D modeling images under different lighting. Step (b) (S20) is a preprocessing process for establishing various learning datasets, and converts the 3D modeling image to 2D by applying different lighting thereto, which is to perform accurate learning on the relationship between RGB and depth. Further, in the step (b), a 2D modeling image of an antagonist tooth or a margin line image of the abutment tooth may be included in the learning dataset.

In this case, in step (b) (S20), a 2D image may be generated with 4 channels of an RGB image (3ch) and an upper surface depth image (1ch) of the tooth.

In summary, in step (b) (S20), all 3D modeling images are converted into 2D images, but, images with shape information and images with depth information are secured into the learning dataset, respectively, by the preprocessing process. In this embodiment, in the process of preprocessing the 3D modeling arrangement of teeth image 101 including the abutment teeth, each RGB image having shape information and a depth image having depth information are secured in 2D. Therefore, in the preprocessing, a 4-channel image conversion process is performed, and this operation is performed in the same way for the crown 3D modeling image 103.

In step (b) (S20), as a preprocessing process, coordinate synchronization of all of the 3D model of the abutment tooth, the 3D model of the prosthesis, and the 3D model of the antagonist tooth is performed. Further, a margin line may be set in this process. Thereafter, in step (b) (S20), rendering is performed as the preprocessing process. A Synchronized RGB-D image is obtained based on the synchronized 3D model.

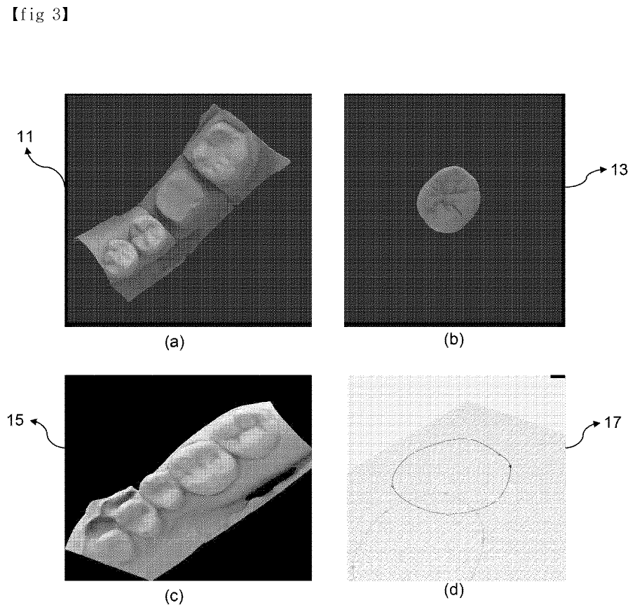

FIG. 3 illustrates a 2D image of a learning dataset obtained by preprocessing the 3D modeling image. Referring to FIG. 3, (a) of FIG. 3 illustrates a 2D modeling image 11 of the abutment tooth, as a preprocessed arrangement of teeth image. (b) of FIG. 3 illustrates a 2D modeling image 13 of the crown as a preprocessed crown image. (c) of FIG. 3 illustrates a 2D modeling image 15 of an antagonist tooth, as a preprocessed antagonist image. (d) of FIG. 3 illustrates a margin line image 17. (a) to (d) of FIG. 3 are classified into the learning dataset for performing first artificial intelligence learning and second artificial intelligence learning, which will be described later.

Step (c) (S30) is a step of performing the first artificial intelligence learning by using the artificial intelligence image conversion algorithm with the abutment tooth image 11 and the crown image having been established into the learning data set as reference data and a crown image generated appropriately to be intubated into the abutment tooth considering a shape and a size of the peripheral tooth of the abutment as a correct answer data. The crown image 13, which is reference data, is data obtained regardless of the type of arrangement of teeth, and a crown image properly designed for the arrangement of teeth image is used as a target of the crown image of the correct answer data.

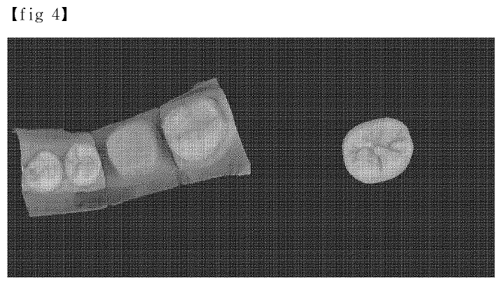

For FIG. 4, drawings related to the first artificial intelligence learning model or the screen being learned will be added.

In step (c) (S30), an artificial intelligence image conversion algorithm of a neural network model utilizing an image encoder decoder may be used. In this embodiment of the artificial intelligence algorithm, the gan algorithm model to which unet is applied may be applied to the first artificial intelligence learning.

For the problem of dealing with images, there is already a good neural network model called CNN. CNN learns to minimize the loss function that informs quality of the result. Although a learning process itself is automated in CNN, there are still many things that need to be manually adjusted in order to produce good results. That is, as it is necessary to present to the CNN what to minimize, it is not suitable as a learning algorithm that self-designs an appropriate shape as in this embodiment. A GAN algorithm has been studied so that the network can reduce the loss according to its goal by itself, and the GAN performs learning on its own so that it cannot distinguish between real and fake, and generates a real clear image. The GAN is suitable for a problem of image conversion that generates an appropriate output image under the condition of an input image, and unet is a type of image encoder decoder neural network. In the unet, information before compression is transmitted from the encoder to the decoder through skip connection during the process in which the encoder compresses information of an image and the decoder converts the information, and thus when applied to GAN, the correlation between the existing image and the generated image can be maintained more clearly. Accordingly, it will be particularly suitable for learning the crown shape according to an arrangement (rgb) of a peripheral arrangement of teeth, the height of the functional cusp (depth), and the shape of the peripheral tooth.

In the step (c) (S30), learning of a first-stage model with the 2D modeling image 11 of the abutment tooth as reference data and the 2D modeling image 13 of the crown as correct answer data and a second-stage model with a result of the first-stage model as reference data and the 2D modeling image of the crown as correct answer data may be performed by the first artificial intelligence learning.

In summary, the first artificial intelligence learning performed in the step (c) (S30) is a step of designing an appropriate shape of the crown based on the unet.

The appropriate shape means that the size, shape, and position of the peripheral tooth are considered and the occlusal surface with the antagonist teeth is designed to be natural. The first artificial intelligence learning may be performed with a two-stage model in order to express intended performance.

FIG. 4 is a learning screen performed in the first-stage model of the first artificial intelligence learning.

Referring to FIG. 4, learning is performed with the 2D image of the abutment or abutment tooth including a peripheral tooth on the left side as reference data and the image of an actual crown on the right side as correct answer data. Through the first-stage model, the unet learns a crown image having a shape, height, and arrangement suitable for the surrounding environment based on information of the arrangement of teeth around the abutment tooth.

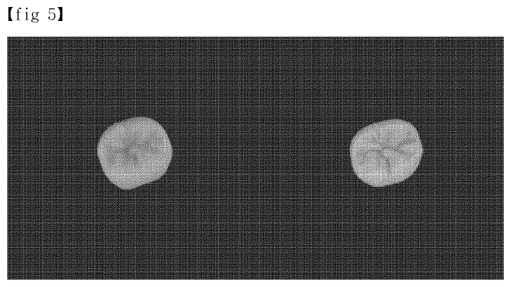

FIG. 5 is a learning screen performed in the second-stage model of the first artificial intelligence learning.

Referring to FIG. 5, in the second-stage model, learning is performed with the image of the crown generated in the first-stage model, the abutment tooth image, the antagonist tooth image, and margin line data as reference data, and an actual crown image as correct answer data. Through the second stage model, the unet learns the natural expression of light and the correlation between RGB and depth.

The reason why this is separately trained is that the similarity between reference data and generated data is required for the discriminator made to derive the loss value to exhibit higher performance in GAN.

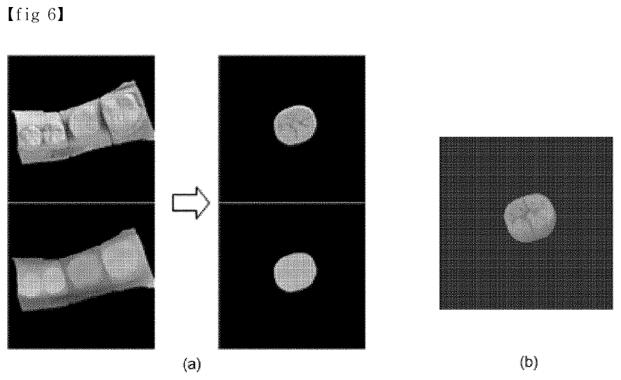

FIG. 6 illustrates the learning screen and learning result of the first artificial intelligence learning. (a) of FIG. 6 illustrates the result of learning from the first-stage model, (b) of FIG. 6 illustrates the result of learning the second-stage model. Referring to FIG. 6, by the first artificial intelligence learning, 2D data of the arrangement of teeth including the abutment tooth and a 2D image of a suitable crown are learned, and the correlation between RGB and depth is also learned. Then, correlation information of RGB and depth expands the image of the crown to 3D through second artificial intelligence learning.

Step (d) (S40) is a step of performing the second artificial intelligence learning for extracting a 3D learning image of the crown in which a volume is assigned to the 2D learning image of the crown by using the artificial intelligence image conversion algorithm on the basis of shape information and depth information of the 2D learning image of the crown and the 2D learning image of the abutment extracted by the first artificial intelligence learning.

In the step (d) (S40), a three-dimensional reconstruction algorithm for composing a 2D image into a 3D image as a neural network model utilizing an image encoder decoder can be applied.

The three-dimensional reconstruction algorithm can be a pixel-aligned implicit function model that implicitly expresses the context of a 2D object associated with the image while locally matching the pixels of the 2D image. Using an image encoder decoder called stacked-hourglass, 3D information is generated based on the shading and depth information of the image, and a 3D model is generated by reinterpreting it as a full-connected layer model according to the coordinates. Unlike voxel representation, this model is memory efficient, and can be implemented in a form that can be generally inferred through learning even in invisible areas.

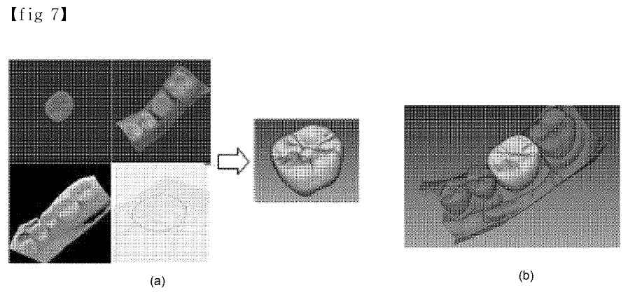

FIG. 7 illustrates a learning screen and learning result of the second artificial intelligence learning. (a) of FIG. 7 illustrates a 3D learning image of a crown constructed by the second artificial intelligence learning, and (b) of FIG. 7 illustrates the result of matching the 3D learning image of the crown to the image of arrangement of teeth.

In step (d) (S40), a 3D learning image of the crown may be generated based on an RGB-D 4-channel image of the abutment tooth and an RGB-D 4-channel image of the crown extracted by the first artificial intelligence learning.

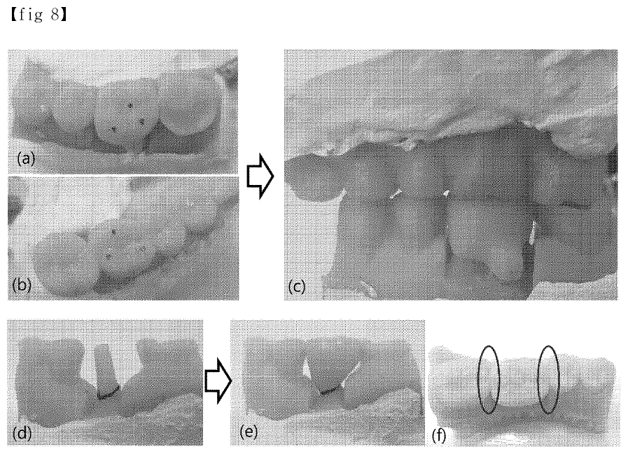

FIG. 8 illustrates a processed product produced as a result of the second artificial intelligence learning.

In this embodiment, in the second artificial intelligence learning performed in step (d) (S40), the upper surface of the crown can be designed to meet the dental occlusion condition when constructing a 3D crown image by learning margin line information of the abutment tooth and occlusal surface information of the crown.

In this embodiment, the artificial intelligence-based application for manufacturing a 3D prosthesis for tooth restoration can be executed by a smartphone, tablet, laptop, or computer having an input means for inputting data, a processing means for processing the input data, and an output means, and

a function (a) (S10) of obtaining an image of arrangement of teeth including an abutment tooth and the 3D modeling image of the crown which is a prosthesis, a function (b) (S20) of preprocessing the 3D modeling image of the abutment tooth, the 3D modeling image of the crown, and a tooth image at a position opposite to the crown into a 2D image so as to establish a learning data set, a function (c) (S30) of performing the first artificial intelligence learning by using an artificial intelligence image conversion algorithm with the abutment tooth image and the crown image having been established into the learning data set as reference data and a crown image generated appropriately to be intubated into the abutment tooth considering a shape and a size of a peripheral tooth of the abutment tooth as a correct answer data, and a function (d) (S20) of performing second artificial intelligence learning for extracting a 3D learning image of a crown in which a volume is assigned to the 2D learning image of the crown from the learning dataset established from the step (b) by using the artificial intelligence image conversion algorithm on the basis of shape information and depth information of the 2D image of the crown, and a 2D image of an occlusal surface of a tooth at a position opposite to the crown can be executed.

In this embodiment, in the (d) function (S40), a 3D image of the crown may be constructed by reflecting information on the occlusal surface of the tooth. Accordingly, in this embodiment, in the function (a) (S10), as input information, not only the image of the arrangement of teeth in which the crown including the abutment tooth is located, but also the tooth image at a position opposite to the crown may be received as an input. The tooth image in the opposite position may be an image of arrangement of teeth in the upper jaw if the crown is located in the lower jaw, or an image of arrangement of teeth in the lower jaw if the crown is located in the upper jaw.

In the function (b) (S20), a learning data set may be established by preprocessing the tooth image at a position opposite to the crown into a 2D image. Here, the tooth image may be an image of an occlusal surface. The image of the occlusal surface reflects the depth information, and the most protruding and most recessed areas on the upper surface of the tooth can be designated as features for learning.

In the function (d) (S40), the occlusal surface can be constructed using the 2D image constructed from the learning dataset of the crown. The image of the crown used here may be an image obtained by preprocessing the 3D modeling image of the crown into 2D. On the other hand, in another embodiment, constructing the occlusal surface in function (d) (S40) may be based on the 2D image of the crown on which the function (c) (S30) has been performed. That is, the function (d) (S40) may be performed based on the 2D image of the crown established by the first artificial intelligence learning and the 2D image of the tooth at a position opposite to the crown. This case is the same as described in the embodiment of FIGS. 1 to 7 described above.

According to the embodiment of FIGS. 1 to 8, the application for manufacturing the 3D prosthesis according to this embodiment includes a function of loading the tooth data of the patient received as an input and a function of executing step (d) (S40), and thus the user can obtain a prosthesis image of 3D crown with a click operation of loading and executing the application when using the application without design work. Function (a) (S10) to function (c) (S30) can be utilized when implementing big data, and the execution controlled by the user may be the function (d) (S40).

As a feature of this embodiment, the user can automatically create an image of a customized prosthesis with just two clicks: a task of loading tooth data of a patient and a task of generating the prosthesis. In this regard, it can be seen from the YouTube (https://youtube/tXNN2cSxG7k) link that the applicant of this application has performed a task of loading tooth data and a task of generating a prosthesis on the artificial intelligence-based application for manufacturing the 3D prosthesis for tooth restoration created by the present applicant.

Referring to FIG. 8, a prosthesis produced by processing a 3D modeling arrangement of teeth formed by executing the function (a) (S10) and the function (d) (S40) according to the present embodiment is illustrated. (a) of FIG. 8 illustrates an arrangement of teeth of the upper jaw, (b) of FIG. 8 illustrates an arrangement of teeth of the lower jaw, and 8(c) of FIG. 8 illustrates a state in which the crown prosthesis formed on the lower jaw is accurately occluded with the upper jaw. (d) of FIG. 8 illustrates the margin line, (e) of FIG. 8 illustrates a state of the crown manufactured according to the margin line, and (f) of FIG. 8 illustrates a state in which junction surfaces of the finally formed crown and the left and right teeth are accurately configured.

Although the present invention has been described in detail through representative examples above, those skilled in the art to which the present invention pertains will understand that various modifications can be made to the embodiments described above without departing from the scope of the present invention. Therefore, the scope of the present invention should not be determined by being limited to the described embodiments, and should be determined by all changes or modifications derived from the concepts equivalent to the scope of the claims as well as the claims to be described later.
30: server
10: 3D scanner
5: patient's teeth
101: 3D modeling image of arrangement of teeth
103: 3D modeling image of crown
11: 2D modeling image of arrangement of teeth including abutment tooth
13: 2D modeling image of crown
15: 2D modeling image of antagonist tooth
17: margin line image

### INDUSTRIAL APPLICABILITY

According to the present invention, manual work by an operator is not required in designing a prosthesis, so that time and cost can be reduced when manufacturing the prosthesis.

1. An artificial intelligence-based method for manufacturing a prosthesis for tooth restoration, the method comprising the steps of:
(a) obtaining an image of arrangement of teeth including an abutment tooth and a 3D modeling image of a crown which is a prosthesis;
(b) preprocessing the 3D modeling image of the abutment tooth and the 3D modeling image of the crown into a 2D image so as to establish a learning dataset;
(c) performing first artificial intelligence learning by using an artificial intelligence image conversion algorithm with the abutment tooth image and the crown image having been established into the learning dataset as reference data and a crown image generated appropriately to be intubated into the abutment tooth considering a shape and a size of a peripheral tooth of the abutment tooth as a correct answer data; and
(d) performing second artificial intelligence learning for extracting a 3D learning image of a crown in which a volume is assigned to the 2D learning image of the crown by using the artificial intelligence image conversion algorithm on the basis of shape information and depth information of the 2D learning image of the crown and the 2D learning image of the abutment tooth extracted by the first artificial intelligence learning.

2. The method of claim 1, wherein
in the step (a),
an arrangement of teeth image including at least one tooth on the left and right of the abutment tooth is used as a modeling target as the image of the arrangement of teeth for which 3D modeling is performed.

3. The method of claim 1, wherein
in the step (b),
the 3D modeling image of the abutment tooth and the 3D modeling image of the crown is pre-processed into a 2D image obtained by photographing cross-sections of the 3D modeling images with a certain angle as a reference.

4. The method of claim 1, wherein
in the step (b),
the 3D modeling image of the abutment tooth and the 3D modeling image of the crown is pre-processed into a 2D image obtained by photographing the 3D modeling images under different lighting.

5. The method of claim 1, wherein
in the step (b),
a 2D modeling image of an antagonist tooth or a margin line image of the abutment tooth may be included in the learning dataset.

6. The method of claim 1, wherein
in the step (b),
a 2D image is generated with 4 channels of an RGB image (3ch) and an upper surface depth image (1ch) of a tooth.

7. The method of claim 1, wherein
in the step (c),
an artificial intelligence image conversion algorithm of a neural network model utilizing an image encoder decoder is used.

8. The method of claim 1, wherein
in the step (c),
learning of a first-stage model with the 2D modeling image of the abutment tooth as reference data and the 2D modeling image of the crown as correct answer data, and
a second-stage model with a result of the first-stage model as reference data and the 2D modeling image of the crown as correct answer data
are performed by the first artificial intelligence learning.

9. The method of claim 1, wherein
in the step (d),
a three-dimensional reconstruction algorithm for composing a 2D image into a 3D image is applied as a neural network model utilizing an image encoder decoder.

10. The method of claim 1, wherein
in the step (d),
a 3D learning image of the crown is generated based on an RGB-D 4-channel image of the abutment tooth and an RGB-D 4-channel image of the crown extracted by the first artificial intelligence learning.

11. An artificial intelligence-based application for manufacturing a 3D prosthesis for tooth restoration, the application being stored in a medium in order to cause a smartphone, tablet, laptop, or computer having an input means for inputting data, a processing means for processing the input data, and an output means to execute the functions of:
(a) obtaining an image of arrangement of teeth including an abutment tooth and a 3D modeling image of a crown which is a prosthesis;
(b) preprocessing the 3D modeling image of the abutment tooth and the 3D modeling image of the crown into a 2D image so as to establish a learning dataset;
(c) performing first artificial intelligence learning by using an artificial intelligence image conversion algorithm with the abutment tooth image and the crown image having been established into the learning dataset as reference data and a crown image generated appropriately to be intubated into the abutment tooth considering a shape and a size of a peripheral tooth of the abutment tooth as a correct answer data; and
(d) performing second artificial intelligence learning for extracting a 3D learning image of a crown in which a volume is assigned to the 2D learning image of the crown by using the artificial intelligence image conversion algorithm on the basis of shape information and depth information of the 2D learning image of the crown and the 2D learning image of the abutment tooth extracted by the first artificial intelligence learning.

12. An artificial intelligence-based application for manufacturing a 3D prosthesis for tooth restoration, the application being stored in a medium in order to cause a smartphone, tablet, laptop, or computer having an input means for inputting data, a processing means for processing the input data, and an output means to execute the functions of:
(a) obtaining an image of arrangement of teeth including an abutment tooth and a 3D modeling image of a crown which is a prosthesis;
(b) preprocessing the 3D modeling image of the abutment tooth, the 3D modeling image of the crown, and a tooth image at a position opposite to the crown into a 2D image so as to establish a learning dataset;
(c) performing first artificial intelligence learning by using an artificial intelligence image conversion algorithm with the abutment tooth image and the crown image having been established into the learning dataset as reference data and a crown image generated appropriately to be intubated into the abutment tooth considering a shape and a size of a peripheral tooth of the abutment tooth as a correct answer data; and
(d) performing second artificial intelligence learning for extracting a 3D learning image of a crown in which a volume is assigned to the 2D learning image of the crown from the learning dataset established from the step (b) by using the artificial intelligence image conversion algorithm on the basis of shape information and depth information of the 2D image of the crown and a 2D image of an occlusal surface of a tooth at a position opposite to the crown.

13. The application of claim 12, further comprising:
a function of loading tooth data of a patient received as an input and a function of executing the step (d), wherein
the user obtains a 3D crown prosthesis image with a click operation of loading and execution when using an application without design work.
